(19) United States
(12) Patent Application Publication
(10) Pub. No.: US 2023/0270755 A1
(43) Pub. Date: Aug. 31, 2023

(54) COMBINATION OF MEDICATIONS IN A PARENTERAL DOSAGE FORM FOR LONG-TERM ANAESTHESIA

(71) Applicant: PHARMAFINA ILAC SATIS VE PAZARLAMA ANONIM SIRKETI, Istanbul (TR)

(72) Inventor: Ibrahim TURAN, Istanbul (TR)

(73) Assignee: PHARMAFINA ILAC SATIS VE PAZARLAMA ANONIM SIRKETI, Istanbul (TR)

(21) Appl. No.: 18/017,860

(22) PCT Filed: Jul. 25, 2020
(86) PCT No.: PCT/TR2020/050655

§ 371 Date: Jan. 25, 2023

(51) Int. Cl. A61K 31/573; A61K 31/445; A61K 31/4168; A61K 9/00; A61K 9/08; A61P 23/00

(52) U.S. Cl. CPC A61K 31/573; A61K 31/445; A61K 31/4168; A61K 9/0019; A61K 9/08; A61P 23/00

(57) ABSTRACT

A combination therapy for controlling and alleviating pain is provided. A long-acting pharmaceutical combination in a parenteral dosage form can be administered before or after a surgery without causing motor nerve blockade. The pharmaceutical combination according to the present invention includes ropivacaine, clonidine, betamethasone, or pharmaceutically acceptable salts and hydrates thereof.

### CROSS REFERENCE TO THE RELATED APPLICATIONS

This application is the national phase entry of International Application No. PCT/TR2020/050655, filed on Jul. 25, 2020, the entire contents of which are incorporated herein by reference.

### TECHNICAL FIELD

The present invention relates to a novel combination therapy for controlling and alleviating pain. More particularly, the invention pertains to a novel long-acting pharmaceutical combination in a parenteral dosage form which can be administered before or after surgery. The pharmaceutical combination according to the present invention comprises ropivacaine, clonidine, betamethasone, or pharmaceutically acceptable salts and hydrates thereof.

### BACKGROUND

Local anesthetics are drugs that block the sensation of pain in the region where they are administered, by reversibly blocking the sodium channels of nerve fibers, thereby inhibiting the conduction of nerve pulses. Local anesthetics are present in the market in different pharmaceutical dosage forms including solution, cream, gel, liposomal capsule, tablet and spray formulations. A common problem associated with local anesthetics is that their half life is not long enough to provide a longer action and more comfort for the patient. Most local anesthetics lose their sensible effect within about two to six hours which is generally not enough especially in post-operative conditions. One another problem of the conventional local anesthetics is that their combinations with other drugs are not well tolerated causing adverse effects. Moreover, their action is mostly dose dependent and higher doses are required for pain management in longer periods.

US 2009/220611 A discloses a microparticulate system with modified release comprising an active principle selected from a list containing several of active ingredients such as anesthetics, analgesics, anticancer agents etc. Microparticles are said to be advantageous as they are well tolerated and they can be obtained easily and economically.

Likewise, US 2006/018933 A provides a modified release dosage form comprising a high solubility active ingredient as well as micro-matrix particles, hydrophobic release controlling agent and a coating. Again there are long lists of alternative active ingredients including anesthetics, analgesics and steroids which can be formulated with modified release. WO 11139595 A2 defines a specific device for administering a therapeutic agent into spinal column wherein the therapeutic agent is selected from a certain list including analgesic and anti-inflammatory agents.

WO 9851290 A2 discloses local anesthetic formulations for causing nerve blockade comprising an agent selected from the group consisting of local anesthetics, vasoconstrictors, adrenergic drugs, vanilloids, amphiphilic solvents, lipophilic solvents, glucocorticoids, and controlled or prolonged release formulations.

These formulations of the prior art, however, are inadequate to provide the desired comfort for the patient especially in post-operative conditions. There is no specific therapy provided as a local anesthetic which may have a longer action while being more tolerable even if higher doses are used in the formulations. It is hard to formulate local anesthetics having more than two active ingredients as they can be incompatible and intolerable. Moreover, combinations of local anesthetics with analgesic agents may cause blocking of motor neurons which may be inhibiting patient's abilities and motion. Local anesthetics are also not suitable for use in anesthesia of surgery involving severe tissue damages. Therefore, there is still an unmet need in state of the art to provide improved local anesthetics for increasing patient's comfort. These problems are presently solved with a pharmaceutical combination comprising ropivacaine, clonidine, betamethasone, or pharmaceutically acceptable salts and hydrates thereof, as defined in the appended claims.

With the pharmaceutical combination of the present invention, day surgery of certain operations become applicable as the patients don't require overnight hospital stay. It is also possible to reduce complications of general anesthesia as its use is presently eliminated. Patients don't need an uptake of heavy analgesics and opioids that cause addiction. Moreover, the novel combinations of the present invention are stable and exhibit low toxicity due to the chemical nature and specific doses of the active ingredients used in the combination.

### SUMMARY

The present invention relates to a pharmaceutical combination in a parenteral dosage form comprising the following ingredients, or pharmaceutically acceptable salts and hydrates thereof:

| Ingredient | Amount |
|---|---|
| Ropivacaine | 10 mg to 200 mg |
| Clonidine | 2.5 μg to 100 μg |
| Betamethasone | 1 mg to 10 mg |

As seen above, the local anesthetic agent according to the present invention is ropivacaine, or pharmaceutical salts and hydrates thereof. More preferably, the local anesthetic agent is hydrochloride salt of ropivacaine, and more preferably is ropivacaine hydrochloride monohydrate. S-enantiomer of ropivacaine is the preferred form of the active ingredient. The dose of ropivacaine is preferably about 25 mg.

Clonidine, as used in the novel combinations of the present invention is preferably clonidine hydrochloride. The dose of Clonidine is preferably about 75 μg.

The corticosteroid agent according to the present invention, namely betamethasone or pharmaceutically acceptable salts thereof is found to be more advantageous as compared to other corticosteroids such as dexamethasone and beclometasone. The dose of betamethasone is preferably about 2 mg.

Therefore, in a preferred embodiment, the combination of the present invention comprises the following ingredients:

| Ingredient | Concentration |
|---|---|
| Ropivacaine | 1 to 20 mg/ml |
| Clonidine | 5 to 100 μg/ml |
| Betamethasone | 0.1 to 10 mg/ml |

In a more preferred embodiment, the combination of the present invention comprises the following ingredients:

| Ingredient | Amount |
|---|---|
| Ropivacaine hydrochloride | equivalent to about 25 mg |
| monohydrate | ropivacaine free base |
| Clonidine hydrochloride | equivalent to about 75 μg |
| | clonidine free base |
| Betamethasone | about 2 mg |
| Saline | about 10 ml |

The pharmaceutical combination according to the present invention is preferably in the form of a composition as a single dose formulated in a pharmaceutically acceptable carrier. Preferably, said pharmaceutically acceptable carrier comprises saline containing sodium and water for injection. The pharmaceutical combination can be provided in the form of an extended release dosage form comprising a carrier selected from the group consisting of liposom, nanocapsule and microcapsule.

The pharmaceutical combination according to the present invention is provided for use in anesthesia and pain control. The anesthesia as mentioned herein may comprise regional anesthesia, spinal anesthesia or epidural anesthesia. The pharmaceutical combination can be administered with injection or infusion via intradermal, subcuteneous, intramuscular, intraosseous, intraperitoneal, intravenous or intravertebral route. Said pharmaceutical combination provides long time local anesthesia which can be used for all types of surgery. But it is particularly useful in post-operative pain control in surgery (i.e. orthopedic surgery).

### DETAILED DESCRIPTION OF THE EMBODIMENTS

The local anesthetic combination of the present invention advantageously contains at least three different types of active ingredients, namely ropivacaine, clonidine, betamethasone, or pharmaceutically acceptable salts and hydrates thereof which are found to be well tolerable and surprisingly having a longer action in pain control without blocking motor nerves, and therefore without causing inhibition of patient's motion and abilities. The novel combination according to the present invention is found to be advantageous especially in controlling pain of orthopedy patients and more particularly post-operative pain of orthopedical surgery.

Ropivacaine, as used in the present invention is an anesthetic compounds conventionally known with an amide group.

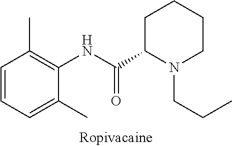 [SMILES: CCCN1CCCC[C@H]1C(=O)Nc1c(C)cccc1C]

Ropivacaine is preferably used as the hydrochloride salt and S-enantiomer thereof for acute pain management and surgical anesthesia. Ropivacaine administered intravenously has the mean half-life of the initial phase of 14 minutes, followed by a slower phase with a mean absorption t_{1/2} of approximately 4.2 hours (Kuthiala et al., Ropivacaine: A review of its pharmacology and clinical use, Indian J Anaesth. 2011 Mar-Apr; 55(2): 104-110). Therefore, ropivacaine loses its sensible effect within about 4 hours and additional doses are required for a longer action.

Clonidine is an agent generally used for lowering blood pressure and alleviating certain kinds of pain. Clonidine has the general formula as follows:

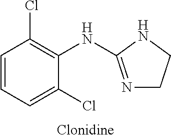

In the context of the present invention, clonidine is preferably used as the hydrochloride salt thereof. Clonidine limits the blood flow and increases duration of action of anesthesia by stimulating α₂ receptors.

Betamethasone is a corticosteroid agent known in the art with the general formula:

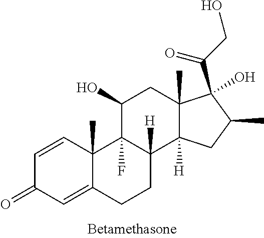

The novel combination of the present invention is particularly found to be increasing patient's comfort as it provides a longer action in pain control without blocking motor nerves but decreasing only sensation.

Therefore, according to an aspect, the present provides a pharmaceutical combination in a parenteral dosage form comprising the foregoing active ingredients. Said combination can be administered simultaneously or concomitantly for the management of pain in a subject. More preferably, said combination is provided as a “pharmaceutical composition” in a parenteral dosage form which is prepared by mixing of said active ingredients in a pharmaceutically acceptable carrier. The parenteral combination of the present invention can be provided in a specific dosage form suitable for administration via intradermal, subcuteneous, intramuscular, intraosseous, intraperitoneal, intravenous or intravertebral route. It is particularly useful in post-operative pain control in surgery, more preferably in post-operative pain control in orthopedic surgery.

In another aspect, the present invention provides novel use of the foregoing combination in surgical anesthesia comprising administering said combination to a patient before, during or after the surgical operation. Said administration can, for instance, be via intravenous infusion or subcutenous route, and more particularly via infiltration anesthesia or epidural administration. The combination according to the present invention is found to be successful especially for the pain management after osteotomy (bone incision) which is generally painful for a longer term as compared to other surgical operations.

In a further aspect, the present invention provides novel use of the foregoing combination in acute pain management comprising administering said combination to a patient via parenteral route as mentioned above via, for instance, continuous infusion or intermittent injections.

In preferred embodiments, the concentration of ropivacaine in the overall volume of the injected solution ranges from 1 to 20 mg/ml, more preferably 2.5 to 5 mg/ml, and most preferably of about 2.5 mg/ml. Each dose may contain 10 mg to 200 mg, and more preferably about 25 mg of local anesthetic which is sufficient for obtaining the desired effect.

Clonidine, as used in the present invention, can be provided with a concentration ranging from 5 to 100 μg/ml, more preferably from 5 to 10 μg/ml. Each dose may contain 2.5 μg to 100 μg, and more preferably about 75 μg of clonidine. These dose values are generally lower than the conventional single dose values available in the market. Therefore, the present invention is advantageous in that the desired therapeutic effect is obtained with lower dose values which in turn decreases adverse effects and increase tolerability of the combination therapy.

Betamethasone according to the present invention can be presented with a concentration ranging from 0.1 to 10 mg/ml, more preferably from 0.2 to 1 mg/ml. Each dose may contain 1 mg to 10 mg, and more preferably 1 mg to 4 mg of betamethasone. Best results are obtained with a dose of about 2 mg.

In the context of the present description, the doses and amounts as mentioned above refer to the active ingredients in free base form. Pharmaceutically acceptable salts and hydrates of said active ingredients contain active molecules with the foregoing amounts and doses equivalent to the free base form.

Preferably, the pharmaceutical combination in parenteral dosage form comprises the following ingredients:
ropivacaine hydrochloride monohydrate,
clonidine hydrochloride,
betamethasone.

Although the pharmaceutical combination according to the present invention provides longer and robust effect in anesthesia for pain control, said combination may be provided in the form of an extended release dosage form comprising a carrier selected from the group consisting of liposom, nanocapsule and microcapsule, which can be desirable especially in severe injuries.

Further aspects and embodiments of the present invention shall be apparent from the appended claims.

Example

Anesthetic preparations of Ropivacain hydrochloride monohydrate were prepared with the following composition by mixing of the active ingredients in saline. The compositions were administered to the patients treated with orthopedic surgery.

Composition

| Ingredient | Amount |
|---|---|
| Ropivacaine hydrochloride monohydrate | 25 mg (2.5 mg/ml) |
| Clonidine hydrochloride | 75 μg (7.5 μg/ml) |
| Betamethasone | 2 mg (0.2 mg/ml) |
| Saline | 10 ml |

All compositions were administered via bolus infusion prior to surgical operation and patients were asked in each hour, during a period of 24 hours, after surgery whether they feel post-operative pain in an uncomfortable level. It has been noted that none of the patients lost their motor abilities and were able to move their respective body parts despite of anesthesia. Patients receiving the composition were able to withstand post-operative pain without receiving extra medication within a 24 hours period and they felt no or moderate during this period.

The composition has surprisingly provided a longer action in post-operative pain management without any loss in functioning of motor nerves, adverse effects and problems in tolerability. Patients felt more comfortable without needing intermittent doses.

It was interestingly found out that the triple combination used for anesthesia of the patients provided very long period without remarkable pain, loss in motor functions and paresis. Without wishing to be bound by a theory, the inventors think that permeability of central nervous system for betamethasone is lower than other corticosteroids such as dexamethasone, and therefore desired effects mentioned above have been improved with the specific corticosteroid and doses of the active ingredients according to the present invention.

What is claimed is:

1. A pharmaceutical combination in a parenteral dosage form comprising the following ingredients or pharmaceutically acceptable salts and hydrates of the following ingredients: 10 mg to 200 mg of ropivacaine, 2.5 μg to 100 μg of clonidine, and 1 mg to 10 mg of betamethasone.

2. The pharmaceutical combination according to claim 1, wherein the pharmaceutical combination is in a form of a composition as a mixture in a pharmaceutically acceptable carrier.

3. The pharmaceutical combination according to claim 1, wherein the pharmaceutical combination comprises ropivacaine hydrochloride in a S-enantiomeric form.

4. The pharmaceutical combination according to claim 3, wherein the pharmaceutical combination comprises a ropivacaine hydrochloride monohydrate.

5. The pharmaceutical combination according to claim 1, wherein the pharmaceutical combination comprises clonidine hydrochloride.

6. The pharmaceutical combination according to claim 2, wherein the pharmaceutically acceptable carrier comprises saline containing sodium and water for an injection.

7. The pharmaceutical combination according to claim 1, comprising the following ingredients: 1 mg to 20 mg of the ropivacaine, 5 μg to 100 μg of the clonidine, and 0.1 mg to 10 mg of the betamethasone.

8. The pharmaceutical combination according to claim 1, comprising the following ingredients: a ropivacaine hydrochloride monohydrate at an amount equivalent to 25 mg of the ropivacaine, clonidine hydrochloride at an amount equivalent to 75 μg of the clonidine, 2 mg of the betamethasone, and 10 ml of saline.

9. The pharmaceutical combination according to claim 1, wherein the pharmaceutical combination is in a form of an extended release dosage form comprising a carrier selected from the group consisting of liposom, nanocapsule, and microcapsule.

10. The pharmaceutical combination in a parenteral dosage form comprising the ingredients according to claim 1 for a use in anesthesia.

11. The pharmaceutical combination for the use according to claim 10, wherein the anesthesia comprises regional anesthesia, spinal anesthesia, or epidural anesthesia.

12. The pharmaceutical combination for the use according to claim 10, wherein the anesthesia comprises an injection or an infusion via an intradermal route, subcuteneous, an intramuscular route, an intraosseous route, an intraperitoneal route, an intravenous route, or an intravertebral route.

13. The pharmaceutical combination for the use according to claim 10, wherein the use is for a post-operative pain control in an orthopedical surgery.

14. The pharmaceutical combination for the use according to claim 13, wherein the orthopedical surgery involves osteotomy.

15. A method for preparing a pharmaceutical composition in a parenteral dosage form comprising mixing the ingredients according to claim 1 in a pharmaceutically acceptable carrier.

16. The pharmaceutical combination according to claim 2, comprising the following ingredients: 1 mg to 20 mg of the ropivacaine, 5 μg to 100 μg of the clonidine, and 0.1 mg to 10 mg of the betamethasone.

17. The pharmaceutical combination according to claim 3, comprising the following ingredients: 1 mg to 20 mg of the ropivacaine, 5 μg to 100 μg of the clonidine, and 0.1 mg to 10 mg of the betamethasone.

18. The pharmaceutical combination according to claim 4, comprising the following ingredients: 1 mg to 20 mg of the ropivacaine, 5 μg to 100 μg of the clonidine, and 0.1 mg to 10 mg of the betamethasone.

19. The pharmaceutical combination according to claim 5, comprising the following ingredients: 1 mg to 20 mg of the ropivacaine, 5 μg to 100 μg of the clonidine, and 0.1 mg to 10 mg of the betamethasone.

20. The pharmaceutical combination according to claim 6, comprising the following ingredients: 1 mg to 20 mg of the ropivacaine, 5 μg to 100 μg of the clonidine, and 0.1 mg to 10 mg of the betamethasone.
